(19) United States
(12) Patent Application Publication
(10) Pub. No.: US 2022/0054099 A1
(43) Pub. Date: Feb. 24, 2022

(54) X-RAY DETECTOR HOLDER STAND

(71) Applicant: Yonghae Han, Seoul (KR)

(72) Inventors: Yonghae Han, Seoul (KR); Jae Dong Rhim, Chungju-si (KR)

(21) Appl. No.: 17/417,457

(22) PCT Filed: Jan. 20, 2020
(86) PCT No.: PCT/KR2020/000971

§ 371 Date: Jun. 23, 2021

(30) Foreign Application Priority Data
Jan. 23, 2019 (KR) 20-2019-0000338
Mar. 12, 2019 (KR) 10-2019-0027850
Mar. 28, 2019 (KR) 20-2019-0001262
Mar. 28, 2019 (KR) 20-2019-0001263

(51) Int. Cl. A61B 6/00; G01T 7/00; F16M 11/18; F16M 11/04; F16M 11/12

(52) U.S. Cl. CPC A61B 6/4405; G01T 7/00; A61B 6/4283; F16M 11/18; F16M 2200/066; F16M 11/128; F16M 2200/047; F16M 2200/028; F16M 2200/021; F16M 11/046

(57) ABSTRACT

An X-ray detector holder stand maintaining the position of a detector is proposed. A pillar erected on a base has a sliding window formed on the front surface of a pillar housing to extend upwards/downwards. A rack is fixed inside the pillar housing to extend perpendicularly. A wire is wound around multiple pulleys to extend perpendicularly to the front/rear side of the rack. A balance weight is coupled to the wire behind the rack inside the pillar housing. An arm includes a sliding block guided along the sliding window, a stopper gear coupled to the sliding block to rotate between a locking position and an unlocking position, an elastic pressurizing means for pressurizing the stopper gear to rotate to the locking position, and a lever means for rotating/operating the stopper gear to the unlocking position. A holder is coupled to an end portion of the arm to enable rotational manipulation.

### TECHNICAL FIELD

The present disclosure relates to an X-ray detector holder stand configured to maintain the position of a detector that is an X-ray device on which an X-ray image is formed.

### BACKGROUND ART

An X-ray detector holder stand is required to maintain a detector at a variety of positions depending on a portion from which an image is to be captured by X-rays or the movement of a patient.

According to these requirements, Japanese Registered Utility Model No. 3189280 (registered on Feb. 12, 2014) proposed an invention regarding a configuration in which a cassette holder 20 is hinge-coupled to the top end of a post 4 through a support arm 11, the post 4 being fixed on a base 2 having casters, as illustrated in FIGS. 1 and 2.

However, when repeatedly used for an extended period of time, the hinge-coupled portion becomes loose due to wear, thereby making it difficult to accurately and uniformly maintain the position of the cassette holder 20 having a relatively heavy weight of 15 kg. Since adjustment in the height or angle is restricted and inaccurate, it is difficult for the disclosed invention to respond to a variety of imaging conditions, such as a patient on a wheelchair, the elderly or the weak, or the position of an affected part. In addition, when the cassette holder 20 is significantly extended forward as illustrated in FIG. 1, there is a risk of inversion.

In addition, since the length of the support arm 11 is fixed at a constant length in FIG. 1, the range of movement of the cassette holder 20 is limited. Thus, it has been frequently difficult for a patient on a wheelchair or a patient with reduced mobility, such as the elderly or the weak, to assume an accurate position for imaging. Sometimes, other patients also have been unable to take a comfortable posture for imaging. For reference, Korean Patent No. 10-0920751 titled “DIGITAL STAND OF X-RAY IMAGING DEVICE FOR SELECTING 3D IMAGING POSITION” (registered on Sep. 30, 2009) proposed a configuration in which a vertical transportation extension support is extensible from and contractible to a vertical transportation reference support. However, this configuration is intended to adjust the height of imaging, and thus, is inappropriate to be applied to adjusting the length of the horizontal arm.

In addition, the cassette holder 20 illustrated in FIG. 1 is fragile in terms of the adjustment in angle and the fixing, and thus, there has been a demand for improvement.

### DISCLOSURE

### Technical Problem

Accordingly, the present disclosure has been made keeping in mind the above problems occurring in the prior art, and an objective of the present disclosure is to provide an X-ray detector holder stand, the position and height of which may be accurately and conveniently adjusted in wider position and angle ranges.

Another objective of the present disclosure is to provide an X-ray detector holder stand having an improved arm extension and contraction structure able to prevent a horizontally extensible and contractible arm from sagging or shaking due to the weight of a cassette.

Another objective of the present disclosure is to provide an X-ray detector holder stand having an improved structure by which the angle of rotation of a holder on which a cassette is mounted may be freely adjusted, the holder may be prevented from sagging or shaking due to the weight of the cassette, and the rotation of the holder may be easily and conveniently adjusted.

### Technical Solution

In order to accomplish at least one of the above objectives, the present disclosure provides an X-ray detector holder stand including: a horizontal base supported on a floor; a post including a post housing fixedly provided upright on the base, with a slide window provided on a front surface of the post housing extending in a top-bottom direction, a rack extending vertically and fixed inside the post housing, a wire wound on a plurality of pulleys arranged in the top-bottom direction within the post housing, the wire extending vertically in front of behind the rack, and a balance scale coupled to the wire behind the rack within the post housing; an arm including a slide block configured to be guided in the top-bottom direction along the slide window by the post, a stopper gear rotatably coupled to the slide block to pivot between a locking position in which the stopper gear is engaged with the rack and an unlocking position in which the stopper gear is disengaged from the rack, an elastic pressing means pressing the stopper gear to rotate to the locking position, and a lever configured to be manipulated to rotate the stopper gear to the unlocking position; and a holder coupled to one end of the arm to be manipulated to rotate in top-bottom and right-left directions.

In addition, in order to accomplish at least one of the above objectives, the present disclosure provides an X-ray detector holder stand including a base supported on a floor, a post provided upright on the base, an arm extending horizontally to be guided in a top-bottom direction along the post, and a holder coupled to a distal end of the arm. The arm may include: an extension block having a plate surface extending horizontally, with an LM guide being fastened integrally to the plate surface; and an end block fitted on the extension block through a rear end, wherein the holder is coupled to a front end of the end block to be rotatable in top-bottom and right-left directions and an LM guide block is slidably coupled to the LM guide and is fastened integrally to a plate surface of the end block, such that the end block is extensible from and contractible to the extension block.

In addition, in order to accomplish at least one of the above objectives, the present disclosure provides an X-ray detector holder stand including a base supported on a floor, a post provided upright on the base, an arm extending horizontally to be guided in a top-bottom direction along the post, and a holder coupled to a distal end of the arm. The holder may be configured to be pivoted in the top-bottom direction by a vertical pivot unit disposed between the holder and a distal end of the arm. The vertical pivot unit may include: a fixing element supporting a fixing pin extending horizontally; a pivot element coupled to the fixing pin to be pivotable in a top-bottom direction so as to pivot between a horizontal angel at which the pivot element is located above the fixing element and a vertical angle located in front of the fixing element; an elastic element axially coupled to the fixing pin, with one end of the elastic element being supported by the fixing element and the other end of the elastic element elastically pressing the pivot element at the horizontal angle; and a pivot angle fixing means for fixing an angle of pivot of the pivot element with respect to the fixing element.

### Advantageous Effects

According to the X-ray detector holder stand according to the present disclosure, even in the case of repetitive use, the position may be reliably fixed due to the rack-gear engagement between the post and the arm, and the height may be adjusted even with a small amount of force due to the balance scale. The arm extending horizontally from the stand facilitates the approaching of the detector to a patient on a wheelchair or the like through the extensible and contractible coupling between the extension block and the end block. In addition, the angle may be adjusted to a wider angle range through the multi-stage horizontal rotation and the multi-stage vertical rotation of the holder and the self-multi-stage rotation of the holder plate. Since the holder plate is provided with the extensible and contractible extension plate, applicability to detectors having a variety of sizes may be obtained. The base of the stand is provided as a plurality of arm plates present on the same plane, thereby being able to prevent the possibility of conduction while having a smaller volume.

In addition, according to the X-ray detector holder stand according to the present disclosure, the horizontally extensible and contractible configuration of the arm may significantly increase the range of movement of the holder, thereby making it easy to appropriately set an imaging position of a patient with reduced mobility. In addition, since the slide coupling structure of the LM guide on the extension block side and the LM guide block on the end block side is provided as a structure for extending and contracting the arm, accurate slide movement between the two sides may be realized. Thus, even when the arm is extended, sagging and shaking due to the weight of the holder side cassette may be prevented. When the extension and contraction of the arm is adjusted, smooth slide movement unique to the LM guide may be realized.

In addition, according to the X-ray detector holder stand according to the present disclosure, the angle of pivoting of the holder in a top-bottom direction may be adjusted using the pivot angle fixing means, such as a combination of the plurality of ball plungers and the plurality of angle adjustment holes. The elastic element elastically pressing the pivot unit to pivot upward may prevent the holder on which the cassette is mounted from suddenly pivoting and protect the holder from resultant impact. Thus, the angle of pivoting may be safely adjusted while being freely adjusted with a small amount of force. In addition, the support force of the elastic unit may prevent the holder with the cassette mounted thereon from sagging or shaking due to the weight of the holder and the cassette.

### DESCRIPTION OF DRAWINGS

FIGS. 1 and 2 are a side view and a plan view illustrating an X-ray detector holder stand of the related art;

FIG. 3 is a perspective view illustrating an X-ray detector holder stand according to a first embodiment of the present disclosure;

FIG. 4 is a side view illustrating the extension and contraction and vertical sliding of the arm and vertical rotation of the holder in the X-ray detector holder stand illustrated in FIG. 3;

FIG. 5 is a plan view illustrating the horizontal movement of the holder in the X-ray detector holder stand illustrated in FIG. 3;

FIG. 6 is a plan view illustrating the self-rotation of the holder and the operation of the extension plate in the X-ray detector holder stand illustrated in FIG. 3;

FIG. 7 is a partially enlarged plan view illustrating the arm and the inside thereof in the X-ray detector holder stand illustrated in FIG. 3 in order to show the extensible and contractible configuration of the arm;

FIG. 8 is a view illustrating the internal configuration and partially enlarged components in the X-ray detector holder stand illustrated in FIG. 3 in order to show a coupling relationship by which the height of the arm with respect to the post may be adjusted;

FIG. 9 is a perspective view illustrating an X-ray detector holder stand according to a second embodiment of the present disclosure;

FIGS. 10 and 11 are front and bottom enlarged views illustrating the arm of the X-ray detector holder stand illustrated in FIG. 9;

FIG. 12 is a cross-sectional view taken along the line A-A in FIG. 10;

FIG. 13 is a cross-sectional view taken along the line B-B in FIG. 10;

FIG. 14 is a plan view illustrating the X-ray detector holder stand illustrated in FIG. 9;

FIGS. 15 and 16 are front views illustrating the extension and contraction of the arm illustrated in FIG. 10;

FIG. 17 is the front view illustrating the X-ray detector holder stand illustrated in FIG. 9;

FIG. 18 is a plan view illustrating the interior, taken along the line A-A in FIG. 17;

FIG. 19 is a cross-sectional view taken along the line B-B in FIG. 18, rotated 90°;

FIG. 20 is a cross-sectional view taken along the line C-C in FIG. 17;

FIG. 21 is an enlarged view illustrating the interior of the holder in FIG. 17, rotated 90°; and

FIG. 22 is an opened-up view illustrating the interior of FIG. 21.

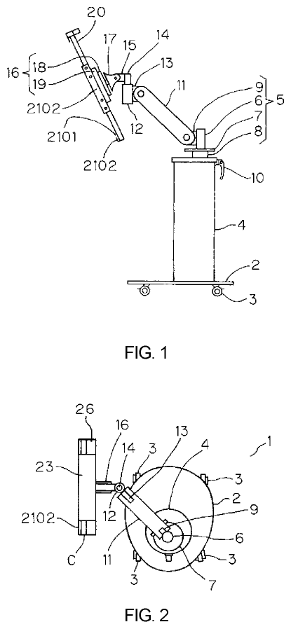

### MODE FOR INVENTION

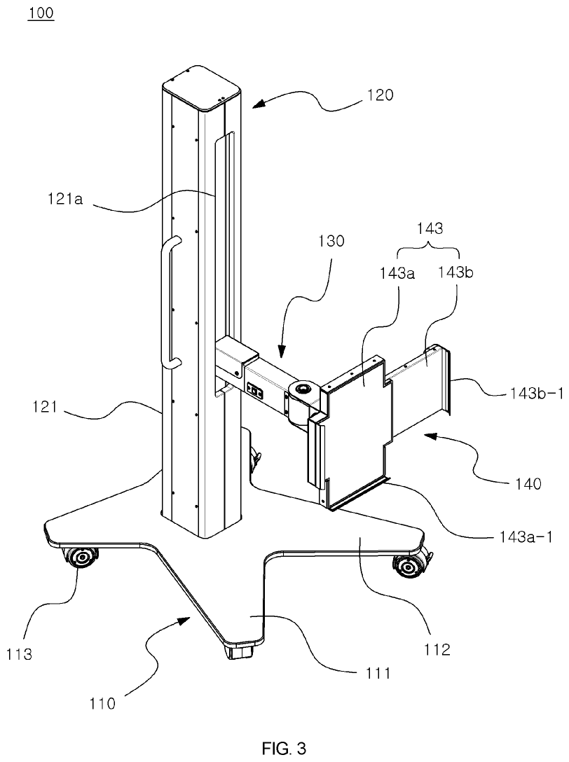

As illustrated in FIG. 3, an X-ray detector holder stand 100 according to a first embodiment of the present disclosure includes: a horizontal base 110 supported on a floor; a post 120 including a post housing 121 fixedly provided upright on the base 110; an arm 130 extending horizontally and configured such that the height thereof is adjustable in a top-bottom direction along a slide window 121a provided on the front surface of the post 120 to extend in a top-bottom direction; and a holder 140 coupled to one end of the arm 130 and configured to be manipulated to rotate in top-bottom and right-left directions.

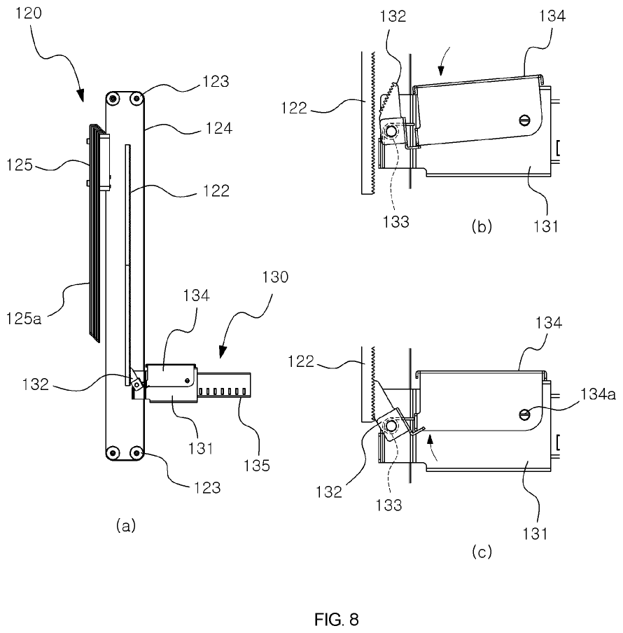

As illustrated in FIG. 8, a rack 122 vertically extending in a top-bottom direction is fixedly disposed within the post housing 121, and a wire 124 is wound on a pair of pulleys 123 fixed to the top and bottom ends within the post housing 121 to extend in a top-bottom direction in front of and behind the rack 122. (The front is right in the figure.) The wire 124 is coupled to a slide block 131 on the arm 130 at a position in front of the rack 122 and is coupled to a balance scale 125 at a position behind the rack 122. The balance scale 125 is provided with weights 125a that may be attached to or detached from the balance scale 125 such that the number of the weights 125a may be appropriately increased or decreased depending on the weight of the holder (140 in FIG. 3) coupled to the balance scale 125 through the arm 130 and a detector (not shown) coupled to the holder.

A stopper gear 132 is rotatably disposed on the slide block 131 to move between a locking position (see c) in which the stopper gear 132 is engaged with the rack 122 and an unlocking position (see b) in which the stopper gear 132 is disengaged from the rack 122. The stopper gear 132 is elastically pressed to the locking position by a spring 133. A block cover 134 covering the slide block 131 from above is coupled to the slide block 131 to be pivotable about a connecting shaft 134a located on one end. The block cover 134 pivots between a pressing position (see b) in which the block cover 134 presses the stopper gear 132 to pivot to the unlocking position and a parking position (see c) in which the block cover 134 is pivoted by the force of the stopper gear 132 biased to rotate toward the locking position (c) due to elastic restorative force of the spring 133.

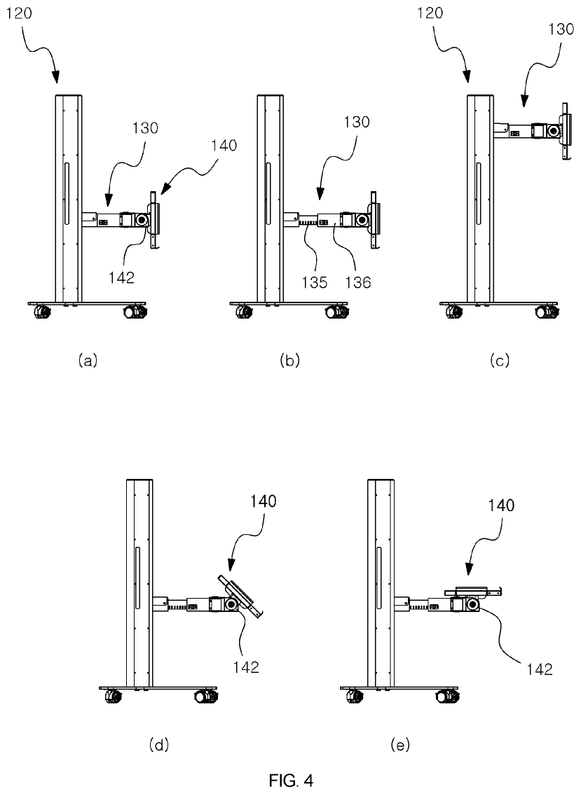

Consequently, as illustrated in (a) and (c) of FIG. 4, the height of the arm 130 is adjustable vertically along the post 120.

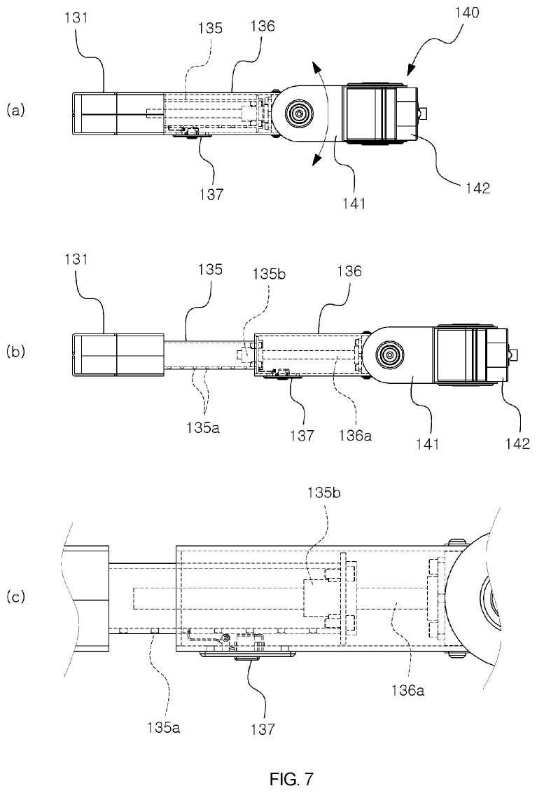

Referring to FIGS. 3 and 7, the arm 130 includes an extension block 135 extending integrally from one end of the slide block 131 and an end block 136 fitted on and coupled to the extension block 135 in an extensible and contractible manner. The extension block 135 has a plurality of length adjustment holes 135a formed in one side surface thereof, and the end block 136 is provided with a switch lever 137 corresponding to the length adjustment holes 135a. The length of the arm 130 may be adjusted, i.e., the arm 130 may be extended and contracted, by manipulating the switch lever 137 to be selectively coupled to the length adjustment holes 135a.

Consequently, as illustrated in FIGS. 4A and 4B, the length of the arm 130 may be adjusted to be increased or decreased.

A shaft 136a extending in the right-left direction along the length of the end block 136 is fixedly disposed within the end block 136, and a flanged ball bearing 135b is disposed on one end within the extension block 135 in a corresponding manner. The shaft 136a and the flanged ball bearing 135b are coupled to each other to guide the movement of the end block 136 with respect to the extension block 135 while preventing the end block 136 from sagging.

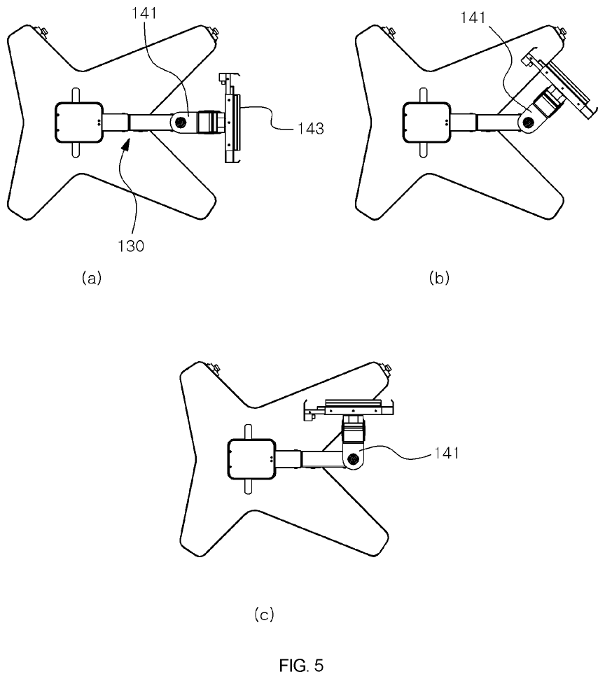

A horizontal joint 141 on the side of the holder 140 is coupled to one end of the end block 136 so as to be rotatable in the right-left direction (see an arrow), and a vertical joint 142 is coupled to one end of the horizontal joint 141 so as to be rotatable in the top-bottom direction (i.e., a direction intersecting the floor). A holder plate 143 as illustrated in FIG. 5 is coupled to one end of the vertical joint 142.

As illustrated in FIG. 5, the holder 140 is configured to perform discontinuous multi-stage rotation at every 45° (b) in the range of a total of 180°, i.e., 90° in the clockwise and counterclockwise directions, with respect to 0°, due to the horizontal joint 141. This multi-stage rotation may be realized by a typical technical implementation, such as a ball-recess coupling relationship due to the pressing of the spring.

As illustrated in FIG. 4, the holder 140 is configured to perform discontinuous multi-stage rotation at every 22.5° (45° in d) in the range of a total of 90° from 90° to 0° in the top direction (not necessary in the bottom direction) due to the vertical joint 142.

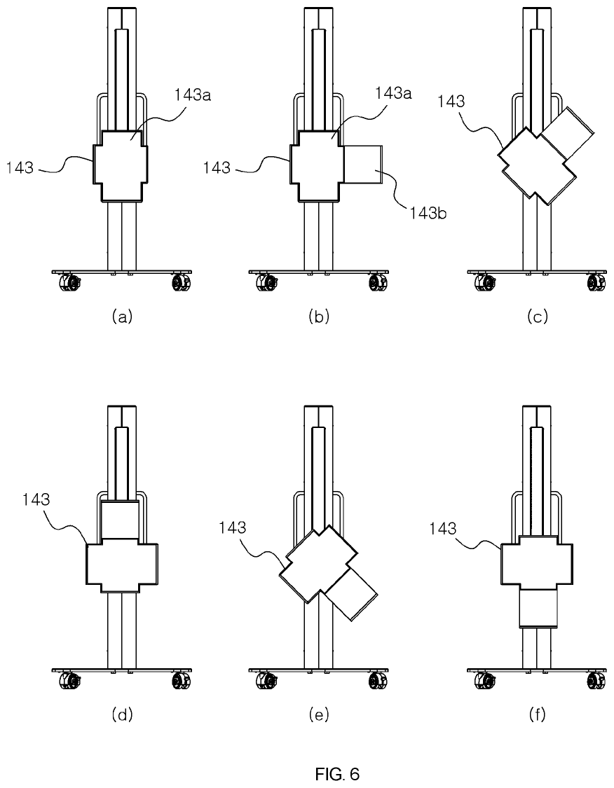

In addition, as illustrated in FIG. 6, the holder plate 143 coupled to one end of the vertical joint 142 illustrated in FIG. 7 is configured to perform discontinuous multi-stage rotation by itself at every 45° in the range of 180° in the clockwise direction with respect to the vertical joint 142 (180° in the sequence of d, c, b, e, and fin FIG. 7).

Referring to FIG. 3, the holder plate 143 may support detectors having a variety of sizes on the body plate 143a and an extension plate 143b coupled to one side of the body plate 143a in an extensible and contractible manner (see a and b in FIG. 6). Support stepped portions 143a-1 and 143b-1 protrude from three sides of the body plate 143a and an extended end of the extension plate 143b to support while being in contact with four sides of a detector.

Referring to FIG. 3 or 5, the base 110 provided as a horizontal flat plate includes a plurality of arm plates 111, 112, and . . . positioned on the same plane and extending radially in different directions and a plurality of casters 113 disposed on bottom ends of the arm plates 111, 112, and . . . . The plurality of arm plates 111, 112, and . . . extending horizontally as described above may prevent the inversion that would otherwise be caused by the high center of gravity due to the relatively heavy detector. In addition, spaces may be formed between the arm plates 111, 112, and . . . , thereby making it easy to approach a patient.

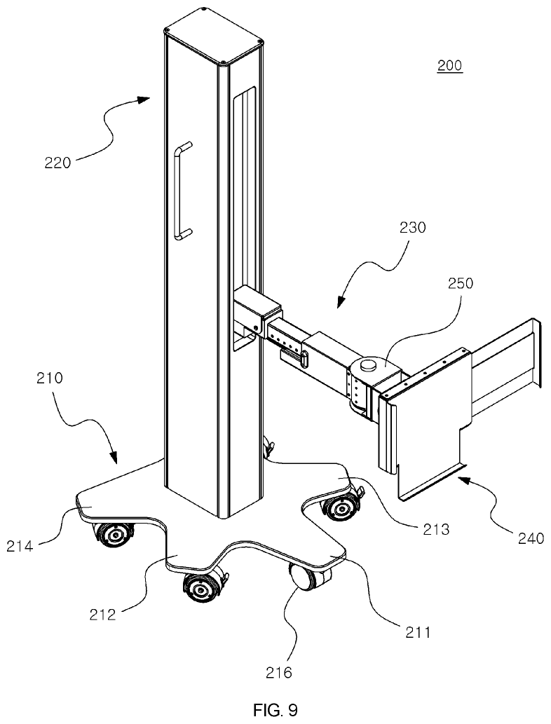

As illustrated in FIG. 9, an X-ray detector holder stand 200 according to a second embodiment of the present disclosure includes: a horizontal base 210 supported on a floor; a post 220 fixedly provided upright on the base 210; an arm 230 extending horizontally and configured such that the height thereof is adjustable in a top-bottom direction along the post 220; and a holder 240 coupled to a front end of the arm 230 and configured to be manipulated to rotate in top-bottom and right-left directions.

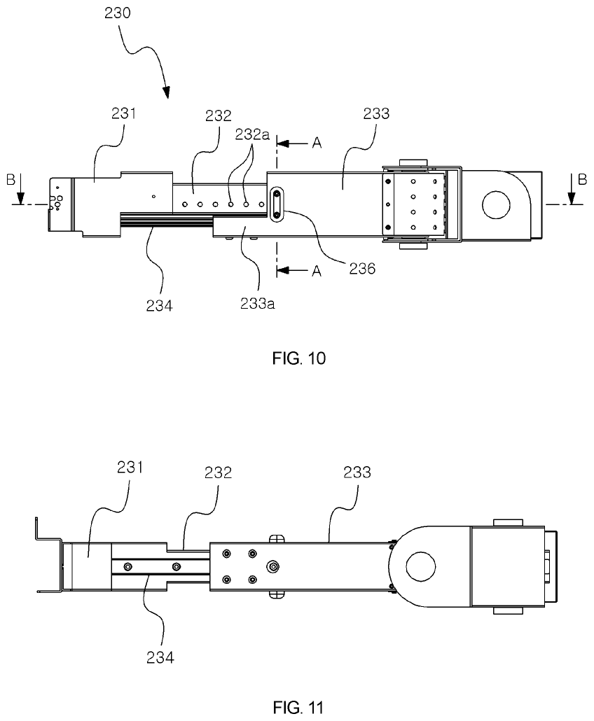

Referring to FIGS. 9 and 10, the arm 230 includes a slide block 231 configured to slide and be fixed in a top-bottom direction along the post 220, an extension block 232 extending integrally from the slide block 231, and an end block 233 fitted on and coupled to the extension block 232 in an extensible and contractible manner.

As illustrated in FIGS. 10 and 11, an LM guide 234 is provided along the plate surface of the extension block 232 extending horizontally and is mounted on and fastened integrally to the bottom surface of the extension block 232.

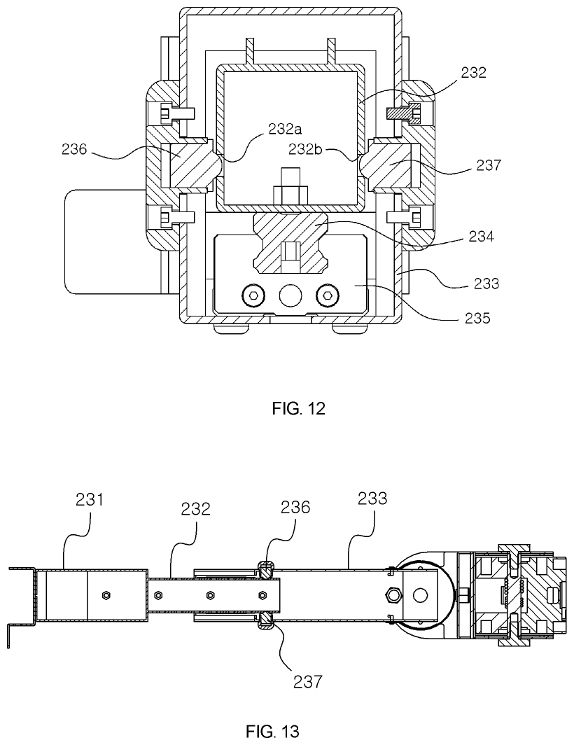

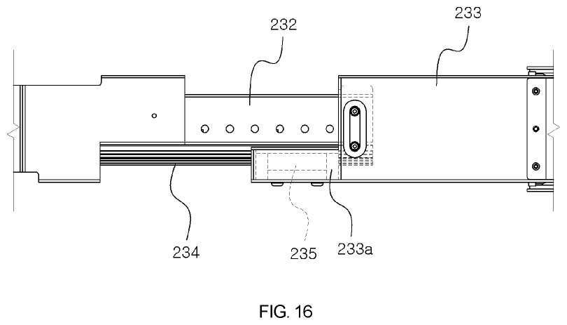

The end block 233 is fitted on the extension block 232 through the rear end of the end block 233. A guide block receiver 233a protruding rearward from the bottom portion of the rear end extends to receive an LM guide block (235 in FIGS. 12 and 15). As illustrated in FIGS. 12 and 15, the LM guide block 235 is mounted on and fastened integrally to the plate surface of the guide block receiver 233a, and in this state, is slidably coupled to the LM guide 234. Due to this coupling between the LM guide 234 and the LM guide block 235, the end block 233 may be provided to be extended from and contracted to the extension block 232, as illustrated in FIGS. 15 and 16.

As illustrated in FIGS. 10 and 12, the extension block 232 has a plurality of length adjustment holes 233a and 233b formed in the right and left side surfaces thereof, and ball plungers 236 and 237 are provided on the left and right side surfaces of the end block 233 in a corresponding manner, When the end block 233 is operated to be extended or contracted, the ball plungers 236 and 237 are selectively coupled to the plurality of length adjustment holes 233a and 233b. In this manner, the end block 233 may be held at the extended or contracted position.

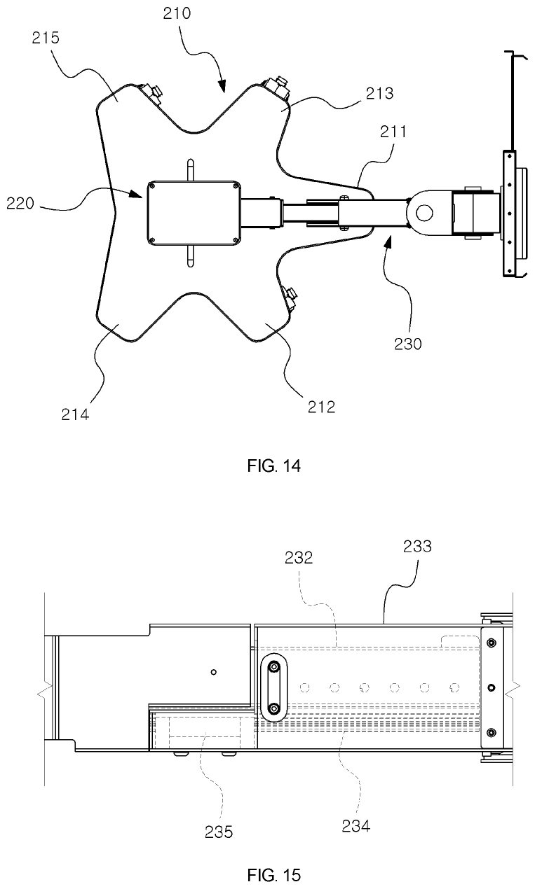

In order to prevent the X-ray detector holder stand 200 from being inverted forward due to the arm 230 having the extensible and contractible end block 233, as illustrated in FIGS. 9 and 14, the base 210 extending horizontally is configured to include a main arm plate 211 and a plurality of auxiliary arm plates 212, 21 3, 214, and 215. The main arm plate 211 extends above or below while being parallel to the arm 230, and the auxiliary arm plates 212, 213, 214, and 215 extend in different directions from a centerline extending in the direction in which the main arm plate 211 extends such that the left auxiliary arm plates are symmetric to the right auxiliary arm plates about the centerline. Casters 216 are disposed on the bottom surfaces of extension ends of the main arm plate 211 and the auxiliary arm plates 212, 213, 214, and 215, respectively.

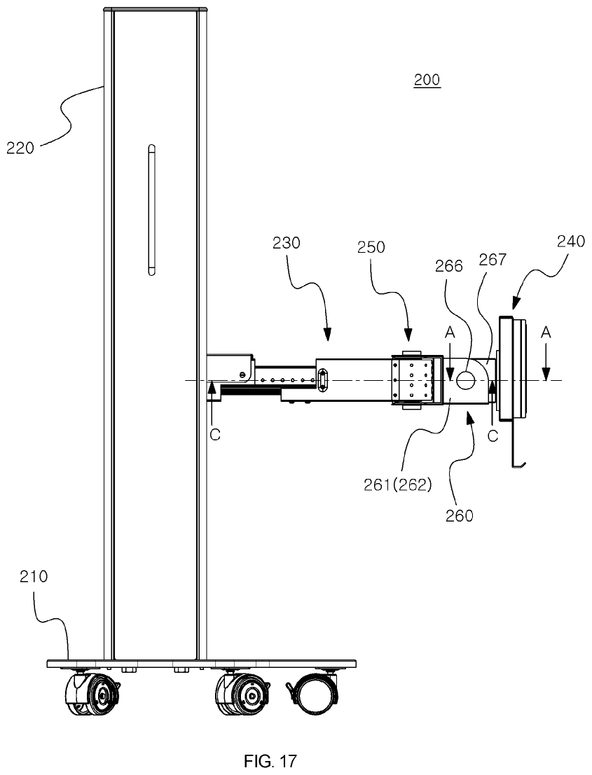

As illustrated in FIG. 17, the X-ray detector holder stand 200 includes: horizontal base 210 supported on the floor; a post 220 fixedly provided upright on the base 210; an arm 230 extending horizontally and configured such that the height thereof is adjustable in a top-bottom direction along the post 220; and a holder 240 coupled to the front end of the arm 230 and configured to be manipulated to rotate in top-bottom and right-left directions.

A horizontal pivot unit 250 and a vertical pivot unit 260 are sequentially disposed between the holder 240 and the arm 230. Thus, the holder 240 is configured to be pivoted in the right-left direction with respect to the arm 230 by the horizontal pivot unit 250 and is pivoted in the top-bottom direction by the vertical pivot unit 260.

As illustrated in FIGS. 17 and 20, the vertical pivot unit 260 includes a fixing bracket 261 as a fixing element including a pair of outer vertical surface portions 262 and 263 and a pair of inner vertical surface portions 264 and 265 and fixing pins 266 extending through the pair of outer vertical surface portions 262 and 263 and the pair of inner vertical surface portions 264 and 265 in the horizontal direction.

In addition, the vertical pivot unit 260 includes a pivot arm 264 as a pivot element that is axially coupled to the fixing pins 266 inside the vertical surface portions 264 and 265 to be pivotable in the top-bottom direction. The pivot arm 264 is configured to pivot between a vertical angle illustrated in FIG. 17 and a horizontal angle illustrated in FIG, 19.

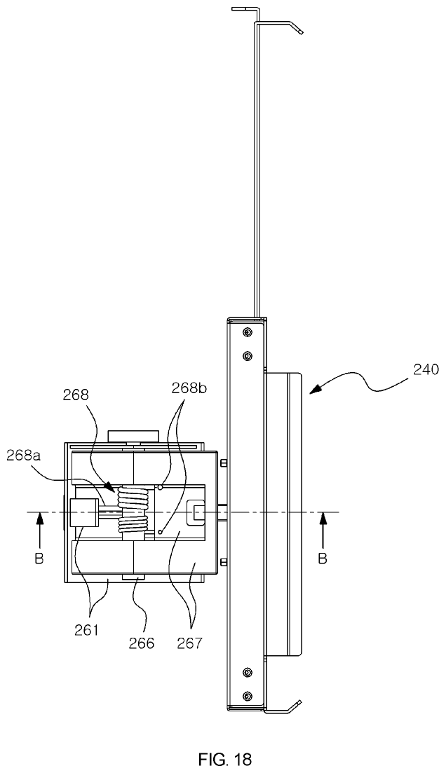

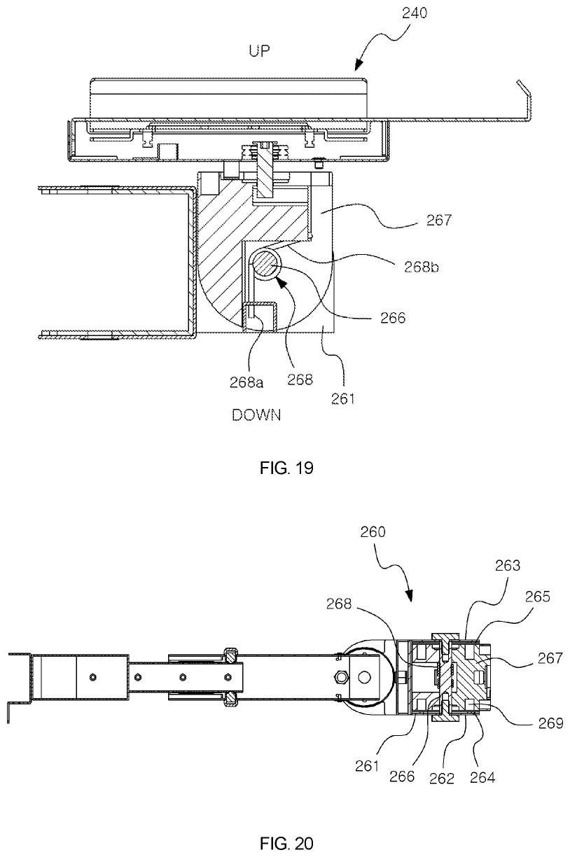

In addition, as illustrated in FIGS. 18 to 20, the vertical pivot unit 260 includes torsion springs 268 as elastic elements. Each of the torsion springs 268 is axially coupled to a corresponding one of the fixing pins 266, with an inner end 268a being supported on the side of the fixing bracket 261, and both outer ends 268b being coupled to the pivot arm 264, so as to elastically press the pivot arm 264 in the direction of the horizontal angle in FIG. 19 (i.e., the counterclockwise direction in the figure). When, at the horizontal angle illustrated in FIG. 19, the holder 240 with a cassette mounted thereon pivots in the clockwise direction, i.e., at the vertical angle, there is a risk that the holder 240 may suddenly pivot due to the weight thereof, thereby being impacted or damaged. During the pivoting, the torsion spring 268 is compressed to apply elastic force in the horizontal direction, i.e., the counterclockwise direction, thereby preventing the holder 240 from suddenly pivoting.

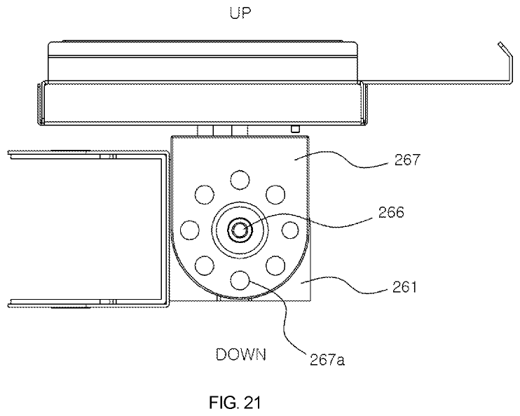

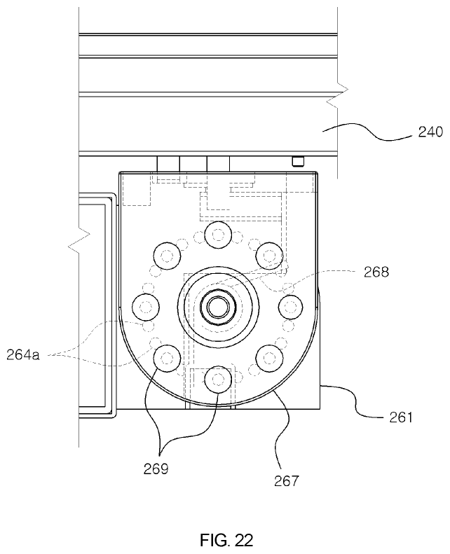

As a pivot angle fixing means for fixing the angle of pivot of the holder 240 pivoting between the horizontal angle (FIG. 8) and the vertical angle (FIG. 6), the vertical pivot unit 260 includes a plurality of ball plungers 269 and a plurality of angle adjustment holes 264a (see FIG. 22) corresponding to the plurality of ball plungers 269. As illustrated in FIGS. 20 and 21, the plurality of ball plungers 269 are fitted into a plurality of fitting holes 267a formed circumferentially in both ends of the pivot arm 264 in the axial direction of the fixing pins 266, and the plurality of angle adjustment holes 264a are formed circumferentially in the pair of inner vertical surface portions 264 and 265 on the side of the fixing bracket 261.

Due to the selective coupling between the plurality of ball plungers 269 and the plurality of angle adjustment holes 264a, the pivot arm 264 may be angle-adjusted and fixed in a multi-stage manner with respect to the fixing bracket 261 as illustrated in FIG. 22

As set forth above, due to the torsion spring 268, the plurality f ball plungers 269, and the plurality of angle adjustment holes 264a, a user may perform the angle adjustment and the fixing by only moving the holder 240 in the pivot direction without any manipulation.

1. An X-ray detector holder stand comprising:
a horizontal base supported on a floor;
a post comprising a post housing fixedly provided upright on the base, with a slide window provided on a front surface of the post housing extending in a top-bottom direction, a rack extending vertically and fixed inside the post housing, a wire wound on a plurality of pulleys arranged in the top-bottom direction within the post housing, the wire extending vertically in front of behind the rack, and a balance scale coupled to the wire behind the rack within the post housing;
an arm comprising a slide block configured to be guided in the top-bottom direction along the slide window by the post, a stopper gear rotatably coupled to the slide block to pivot between a locking position in which the stopper gear is engaged with the rack and an unlocking position in which the stopper gear is disengaged from the rack, an elastic pressing means pressing the stopper gear to rotate to the locking position, and a lever configured to be manipulated to rotate the stopper gear to the unlocking position; and
a holder coupled to one end of the arm to be manipulated to rotate in top-bottom and right-left directions.

2. The X-ray detector holder stand of claim 1, wherein the lever comprises a block cover pivoting between a pressing position in which the block cover is coupled to the slide block so as to be relatively pivotable and presses the stopper gear to pivot to the unlocking position and a parking position in which the block cover is pivoted by rotating force of the stopper gear toward the locking position caused by the elastic pressing means.

3. The X-ray detector holder stand of claim 1, wherein the arm further comprises an extension block extending from one end of the slide block, with a plurality of length adjustment holes being formed in one side surface of the extension block, an end block fitted on the extension block through one end and coupled to the extension block in an extensible and contractible manner, with the holder being coupled to the end block so as to be rotatable in the top-bottom and right-left directions, and a switch lever provided on the end block to be elastically coupled to the length adjustment holes.

4. The X-ray detector holder stand of claim 3, wherein the holder comprises a horizontal joint coupled to one end of the end block to be multi-stage rotatable at every 45° in a range of 180° in the right-left direction, a vertical joint coupled to one end of the horizontal joint to be multi-stage rotatable at every 22.5° in a range of from 0° to 90° in the top-bottom direction, and a holder plate coupled to one end of the vertical joint.

5. The X-ray detector holder stand of claim 4, wherein the holder plate is configured to perform multi-stage self-rotation at every 45° in a range of 180° with respect to the vertical joint.

6. The X-ray detector holder stand of claim 4, wherein the holder plate comprises a body plate shaped as a rectangular flat plate and having three support stepped portions protruding from three sides and an extension plate coupled to a remaining single side of the body plate in an extensible and contractible manner, with a fourth support stepped portion protruding from an extension end of the extension plate.

7. The X-ray detector holder stand of claim 1, wherein the base comprises a plurality of arm plates provided on a single plane and extending circumferentially in different directions and a plurality of casters respectively provided on a corresponding one of bottom portions of the plurality of arm plates.

8. An X-ray detector holder stand comprising:
a base supported on a floor;
a post provided upright on the base;
an arm extending horizontally to be guided in a top-bottom direction along the post; and
a holder coupled to a distal end of the arm,
wherein the arm comprises:
an extension block having a plate surface extending horizontally, with an LM guide being fastened integrally to the plate surface; and
an end block fitted on the extension block through a rear end, wherein the holder is coupled to a front end of the end block to be rotatable in top-bottom and right-left directions and an LM guide block is slidably coupled to the LM guide and is fastened integrally to a plate surface of the end block, such that the end block is extensible from and contractible to the extension block.

9. The X-ray detector holder stand of claim 8, wherein the LM guide is mounted on and fastened integrally to a bottom surface of the extension block, and
wherein the end block comprises a guide block receiver protruding rearward from a bottom portion of the rear end, and the LM guide block is received in the guide block receiver and is integrally fastened to a plate surface of the guide block receiver.

10. The X-ray detector holder stand of claim 8, wherein the extension block has a plurality of length adjustment holes in at least one side surface, and
the end block comprises a ball plunger provided on at least one side surface to be selectively coupled to the plurality of length adjustment holes when the end block is adjusted to extend from or contract to the extension block.

11. The X-ray detector holder stand of claim 10, wherein the plurality of length adjustment holes is provided in right and left side surfaces of the extension block, and the ball plungers are provided on right and left side surfaces of the end block.

12. The X-ray detector holder stand of claim 8, wherein the base comprises a main arm plate extending above or below while being parallel to the arm, a plurality of auxiliary arm plates extending in different directions from a centerline extending in a direction in which the main arm plate extends such that the auxiliary arm plates in the right side are symmetric to the auxiliary arm plates in the left side about the centerline, and a plurality of casters respectively provided on a corresponding one of bottom portions of the main arm plate and the plurality of auxiliary arm plates.

13. An X-ray detector holder stand comprising:
a base supported on a floor;
a post provided upright on the base;
an arm extending horizontally to be guided in a top-bottom direction along the post; and
a holder coupled to a distal end of the arm,
wherein the holder is configured to be pivoted in the top-bottom direction by a vertical pivot unit disposed between the holder and a distal end of the arm, and
wherein the vertical pivot unit comprises:
a fixing element supporting a fixing pin extending horizontally;
a pivot element coupled to the fixing pin to be pivotable in a top-bottom direction so as to pivot between a horizontal angel at which the pivot element is located above the fixing element and a vertical angle located in front of the fixing element;
an elastic element axially coupled to the fixing pin, with one end of the elastic element being supported by the fixing element and the other end of the elastic element elastically pressing the pivot element at the horizontal angle; and
a pivot angle fixing means for fixing an angle of pivot of the pivot element with respect to the fixing element.

14. The X-ray detector holder stand of claim 13, wherein the fixing element comprises a fixing bracket having a pair of vertical surface portions supporting both ends of the fixing pin,
wherein the pivot element comprises a pivot arm axially coupled to the fixing pin inside the pair of vertical surface portions, and
wherein the pivot angle fixing means includes a plurality of angle adjustment holes provided
circumferentially in the pair of vertical surface portions and a plurality of ball plungers
circumferentially disposed on both ends of the pivot arm in an axial direction of the fixing pin to be selectively coupled to the plurality of angle adjustment holes.
